(19) United States
(12) Patent Application Publication
(10) Pub. No.: US 2024/0181269 A1
(43) Pub. Date: Jun. 6, 2024

(54) DEVICE AND METHOD FOR ANTI-TACHYCARDIA PACING

(71) Applicant: Impulse Dynamics NV, Willemstad (CW)

(72) Inventors: Tamir BEN DAVID, Tel-Aviv (IL); David PRUTCHI, Voorhees, NJ (US)

(73) Assignee: Impulse Dynamics NV, Willemstad (CW)

(21) Appl. No.: 18/285,504

(22) PCT Filed: Apr. 4, 2022
(86) PCT No.: PCT/IB2022/053105

§ 371 Date: Oct. 4, 2023

Related U.S. Application Data
(60) Provisional application No. 63/171,097, filed on Apr. 6, 2021.

(51) Int. Cl. A61N 1/39; A61N 1/05; A61N 1/362; A61N 1/365

(52) U.S. Cl. CPC A61N 1/39622; A61N 1/0563; A61N 1/3622; A61N 1/365; A61N 1/3987

(57) ABSTRACT

A method of anti-tachycardia pacing via an implanted cardiac device, comprising: positioning at least one electrode of the implanted cardiac device at an intra-cardiac location; detecting a tachycardia episode; delivering, via the at least one electrode, anti-tachycardia pacing pulses, wherein an anti-tachycardia pacing pulse comprises at least one of: a duration of between 5-10 msec and an amplitude of between 7.5-10V.

### RELATED APPLICATION

This application claims the priority of U.S. Provisional Patent Application No. 63/171,097, filed on 6 Apr. 2021, the contents of which are incorporated by reference as if fully set forth herein in their entirety.

### FIELD AND BACKGROUND OF THE INVENTION

The present invention, in some embodiments thereof, relates to anti-tachycardia (ATP) pacing and, more particularly, but not exclusively, to delivery of ATP pulses having an amplitude and/or duration selected to effectively terminate an arrhythmia.

Patent No. JP2016517771A discloses “Substernal implantable cardioverter defibrillator (ICD) system and method for providing substernal electrical stimulation therapy to treat malignant tachyarrhythmias such as ventricular tachycardia (VT) and ventricular fibrillation (VF) is described. In one embodiment, an implantable cardioverter defibrillator (ICD) system includes an ICD that is implanted in a patient and an implantable medical electrical lead. The lead includes an elongated lead body having a proximal end and a distal portion, a connector at the proximal end of the lead body configured to couple to the ICD, and one or more along the distal portion of the elongated lead body. And more electrodes. The distal portion of the elongate lead body of the lead is implanted substantially within the patient's anterior mediastinum and the ICD is configured to deliver electrical stimulation to the patient's heart using one or more electrodes”.

U.S. Pat. No. 9,031,648B2 discloses “According to one aspect, various methods and apparatus are used for treating a condition of a patient's heart, and for monitoring cardiac operation. In one approach consistent therewith, an electrode arrangement is placed in a right ventricle of the heart. The electrode arrangement is used to capture the myocardium for re-synchronization of the left and right ventricles by providing first and second signal components having opposite polarity on respective electrodes. The electrode arrangement is connected to an implantable CRM device that has the capability of pacing/sensing atrium, pacing/sensing ventricles, and deliver defibrillation therapy from the right side of the heart. The CRM device captures ventricular contractions to treat conduction abnormalities in one or more of the ventricles.”

### SUMMARY OF THE INVENTION

According to an aspect of some embodiments there is provided a method of anti-tachycardia pacing via an implanted cardiac device, comprising:
positioning at least one electrode of the implanted cardiac device at an intra-cardiac location;
detecting a tachycardia episode;
delivering, via the at least one electrode, anti-tachycardia pacing pulses, wherein an anti-tachycardia pacing pulse comprises at least one of: a duration of between 5-10 msec and an amplitude of between 5-10V.

In some embodiments, the intra-cardiac location is one of: the ventricular septum, the left ventricle wall, the right ventricle wall.

In some embodiments, the method comprises positioning at least two electrodes in at least two different intra-cardiac locations.

In some embodiments, the method comprises positioning the at least two electrodes at two different locations within the right ventricle.

In some embodiments, the method comprises positioning the at least two electrodes at two different locations along the ventricular septum.

In some embodiments, the method comprises positioning at least one electrode in the right ventricle and at least one electrode in the left ventricle.

In some embodiments, the method further comprises delivering a defibrillation pulse if the tachycardia episode continues following the delivering of anti-tachycardia pacing pulses.

In some embodiments, detecting is by measuring an R-R interval via the at least one electrode of the implanted cardiac device.

In some embodiments, the anti-tachycardia pulse is a biphasic pulse.

In some embodiments, delivering comprises delivering synchronized anti-tachycardia pacing pulses via the at least two electrodes

In some embodiments, delivering comprises delivering the anti-tachycardia pacing pulses simultaneously via the at least two electrodes.

In some embodiments, delivering comprises delivering the anti-tachycardia pacing pulses with a time delay such that pulses applied via one of the two electrodes are delivered in a predetermined time delay from pulses applied via a second electrode of the two electrodes.

In some embodiments, the predetermined time delay is selected according to a time difference between R-wave occurrences measured by the at least two electrodes at the at least two different cardiac locations during normal cardiac function.

In some embodiments, detecting comprises measuring a heart rate of between 180-250 BPM.

According to an aspect of some embodiments there is provided an implantable cardiac device comprising:
at least one lead electrode configured to contact intra-cardiac tissue;
a housing including circuitry for controlling and activating the at least one lead electrode; and
a pulse generator configured to generate anti-tachycardia pacing pulses to be delivered by the at least one lead electrode, wherein an anti-tachycardia pacing pulse comprises at least one of: a duration of between 5-10 msec and an amplitude of between 5-10V.

In some embodiments, the circuitry comprises a memory which stores a plurality of anti-tachycardia pacing pulse sequences, and a controller configured to implement the delivery of a selected anti-tachycardia pacing pulse sequence.

In some embodiments, the cardiac device is further configured for one or more of: delivering, during normal sinus rhythm, cardiac contractility modulation stimulations having a duration of between 5-10 msec and an amplitude of between 5-10V; and delivering defibrillation shocks.

In some embodiments, the cardiac device comprises at least two lead electrodes configured to contact intra-cardiac tissue.

In some embodiments, the intra-cardiac tissue is one of: the ventricular septum, the left ventricle wall, the right ventricle wall.

In some embodiments, the device comprises at least two lead electrodes configured to be positioned in at least two different intra-cardiac locations.

In some embodiments, the at least two different intra-cardiac locations are within the right ventricle.

In some embodiments, the at least two different intra-cardiac locations are along the ventricular septum.

In some embodiments, the at least two different intra-cardiac locations include the right ventricle and the left ventricle.

In some embodiments, the circuitry is configured to detect a tachycardia episode, and wherein the pulse generator is configured to deliver a defibrillation pulse if a detected tachycardia episode continues following delivery of the anti-tachycardia pacing pulses.

In some embodiments, the circuitry is configured to detect the tachycardia episode by measuring an R-R interval via the at least one lead electrode.

In some embodiments, the circuitry is configured to detect the tachycardia episode when measuring a heart rate of between 180-250 BPM.

In some embodiments, the anti-tachycardia pacing pulse is a biphasic pulse.

In some embodiments, the pulse generator is configured to generate synchronized anti-tachycardia pacing pulses to be delivered via the at least two lead electrodes.

In some embodiments, the pulse generator is configured to generate the anti-tachycardia pacing pulses simultaneously.

In some embodiments, the pulse generator is configured to generate the synchronized anti-tachycardia pacing pulses with a time delay between them such that pulses applied via one of the two lead electrodes are delivered in a predetermined time delay from pulses applied via a second electrode of the at least two lead electrodes.

In some embodiments, the predetermined time delay is selected according to a time difference between R-wave occurrences measured via the at least two electrodes at the at least two different cardiac locations during normal cardiac function.

In some embodiments, the pulse generator is configured to generate anti-tachycardia pacing pulses having different durations selected from between 5-10 msec.

According to an aspect of some embodiments there is provided a method of anti-tachycardia pacing via an implanted cardiac device, comprising:
detecting a tachycardia episode;
delivering a pulse sequence which combines anti-tachycardia pacing pulses having different durations.

In some embodiments, a pulse duration is from between 0.1-10 msec.

In some embodiments, the anti-tachycardia pacing pulses include wide pacing pulses and narrow pacing pulses, wherein a wide pacing pulse comprises a duration of between 2-10 msec and a narrow pacing pulse comprises a duration of between 0.1-5 msec.

In some embodiments, delivering comprises delivering a plurality of narrow pacing pulses followed by a plurality of the wide pacing pulses.

In some embodiments, delivering comprises delivering a sequence including pairs of pulses, wherein in each pair a narrow pacing pulse is followed by a wide pacing pulse, the narrow pacing pulse and the wide pacing pulse delivered within the same cardiac cycle.

In some embodiments, the wide pacing pulse is delivered during a refractory period triggered by the narrow pulse.

In some embodiments, delivering comprises delivering a sequence of pacing pulses which gradually increase in duration.

In some embodiments, each pulse is at least 10% longer than its preceding pulse.

In some embodiments, delivering comprises delivering a sequence of pacing pulses which gradually decrease in duration.

In some embodiments, each pulse is at least 10% shorter than its preceding pulse.

According to an aspect of some embodiments there is provided a method of anti-tachycardia pacing via an implanted cardiac device, comprising:
detecting a tachycardia episode;
delivering, via at least two electrodes positioned in contact with tissue in at least two different intra-cardiac locations, anti-tachycardia pacing pulses.

In some embodiments, the method further comprises measuring, during normal cardiac function, a time difference between R-wave occurrences in the at least two different intra-cardiac locations.

In some embodiments, a time interval between pacing pulses delivered to the two different intra-cardiac locations is set according to the measured time difference.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not 20 intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by 25 software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C are schematic illustrations showing three exemplary configurations of an implantable cardiac device, according to some embodiments;

FIG. 2 is a block diagram of components of an implantable cardiac device, according to some embodiments;

FIG. 3A is a diagram demonstrating anti tachycardia pacing, according to some embodiments;

FIG. 3B schematically illustrates a potential effect of anti-tachycardia pacing pulses on cardiac tissue, according to some embodiments;

FIGS. 3C-D schematically illustrate a field of effect of ATP pulses on cardiac tissue in two examples of ventricular tachycardia conditions, according to some embodiments;

FIG. 4 is a flowchart of a general method for delivering via one or more intra-cardiac electrodes enhanced pacing pulses for treating tachycardia, according to some embodiments;

FIG. 5 is a diagram demonstrating anti tachycardia pacing by delivering synchronized pacing pulses to at least two different cardiac locations, according to some embodiments;

FIG. 6 is a flowchart of a method for timing anti tachycardia pacing delivered to at least two different cardiac locations according to a measured time interval of R-wave occurrence at the two locations, according to some embodiments;

FIG. 7 is a diagram demonstrating anti tachycardia pacing by delivering to at least two different cardiac locations pacing pulses timed according to a time interval of R-wave occurrence at the two locations;

FIG. 8 is a flowchart of a method for delivering a pulse sequence which combines wide pacing pulses and narrow pacing pulses for treating tachycardia, according to some embodiments;

FIG. 9 is a diagram demonstrating anti tachycardia pacing by a first exemplary combination of wide and narrow pacing pulses, according to some embodiments;

FIG. 10 is diagram demonstrating anti tachycardia pacing by a second exemplary combination of wide and narrow pacing pulses, according to some embodiments;

FIG. 11 is diagram demonstrating anti tachycardia pacing by a third exemplary combination of wide and narrow pacing pulses, according to some embodiments; and

FIG. 12 is diagram demonstrating anti tachycardia pacing by a fourth exemplary combination of wide and narrow pacing pulses, according to some embodiments.

### DESCRIPTION OF SPECIFIC EMBODIMENTS OF THE INVENTION

The present invention, in some embodiments thereof, relates to anti-tachycardia (ATP) pacing and, more particularly, but not exclusively, to delivery of ATP pulses having an amplitude and/or a duration selected to effectively terminate an arrhythmia.

A broad aspect of some embodiments relates to anti-tachycardia pacing in which pacing pulses delivered inside the heart are of relatively high power and/or long duration. In some embodiments, ATP pulse parameters (such as pulse amplitude, pulse duration, frequency of applying, timing of applying, number of pulses in a sequence and/or other parameters) are selected to interrupt the tachyarrhythmia and override the natural rhythm. In some embodiments, ATP pulse parameters are selected to affect a relatively large area of cardiac tissue, for example as compared to the tissue area affected by a weaker and/or shorter pulse. Optionally, the ATP pulse is set to capture a larger number of myocardial cells, for example as compared to the number of myocardial cells captured by a weaker and/or shorter pulse.

In some embodiments, the ATP pulses each have an amplitude of, for example, between 7-10V, 5-10 V, 3-8 V or intermediate, higher or lower amplitude and/or a duration of between 5-10 msec, 2-10 msec, 7-12 msec or intermediate, longer or shorter In some embodiments, an upper threshold of the pulse amplitude is selected to be low enough so as to still enable delivery of the signal into the heart tissue itself (to intra-cardiac tissue), without risking damage to the tissue which may be caused by a voltage that is too high. Examples of an upper threshold of the ATP pulse amplitude may include 10V, 9V, 11V, or intermediate, higher or lower voltage.

In some embodiments, the ATP pulse is a bi-phasic pulse. In some embodiments, a plurality of ATP pulses are delivered sequentially, with a time interval of, for example, between 150 msec and 400 msec between subsequent pulses.

In some embodiments, the ATP pulses are delivered via a plurality of lead electrodes (e.g. 1, 2, 3, 4, 5, 6, 8 or intermediate, larger or smaller number of electrodes) which contact intra-cardiac tissue. Optionally, the electrodes contact the inner walls of the heart, for example placed in conductive contact with myocardial tissue.

A potential advantage of anti-tachycardia pacing delivered into the heart with parameters for example as described herein may include interrupting the arrhythmia faster and/or with a smaller amount of pulses (e.g. as compared to use of weaker and/or shorter pulses). Another potential advantage of a stronger and/or longer pulse may include improved conduction of the excitatory signal, for example since the longer and/or stronger signal may be effective to capture myocardial cells located adjacent damaged tissue regions, where the signal conduction could be slowed down or the signal would be caused to pass along an alternative conduction pathway.

An aspect of some embodiments relates to delivering ATP pulses to at least two different intra-cardiac locations. In some embodiments, the ATP pulses are delivered via two or more electrodes of an implanted device, each electrode contacting a different cardiac tissue site. The electrodes may be positioned, for example, at two locations within the right ventricle; at two locations along the ventricular septum; one electrode at the septum and one electrode within the right or left ventricle; one electrode in the right ventricle and one electrode in the left ventricle; and/or other locations.

In some embodiments, ATP pulse sequences delivered via the at least two electrodes are synchronized. Alternatively, ATP pulse sequences are delivered with a predefined delay between the pulses delivered to each of the locations. In some embodiments, the delay is timed according to a time difference between R-wave occurrence at the two locations, optionally measured by the two electrodes.

An aspect of some embodiments relates to an ATP pulse sequence which combines pulses of different durations.

In some embodiments, the pulse sequence combines wide pacing pulses (e.g. having a duration of between 5-10 msec) and narrow pacing pulses (e.g. having a duration of between 0.1-2 msec). In some embodiments, a plurality of narrow pulses are followed by a plurality of wide pulses. In some embodiments, pairs of wide and narrow pulses are delivered, each pair including, for example, a narrow pulse followed by a wide pulse, which is optionally delivered during a refractory period triggered by the narrow pulse.

In some embodiments, the pulses of a sequence gradually increase in duration, for example, each pulse is at least 10%, at least 20% at least 40% or intermediate, larger or smaller percentage longer than its preceding pulse. Alternatively, is some embodiments, the pulses of a sequence gradually decrease in duration, for example, each pulse is at least 10%, at least 20% at least 40% or intermediate, larger or smaller percentage shorter than its preceding pulse.

An aspect of some embodiments relates to an implantable cardiac device configured for delivery of ATP pulses having parameters (e.g. amplitude, duration, frequency) for example as described herein. In some embodiments, the device is provided with powering means (e.g. a battery) and circuitry suitable for generating the relatively strong ATP pulses.

In some embodiments, the device is configured for detection of cardiac episodes and/or cardiac cycle characteristics, for example via one or more lead electrodes of the device (which also deliver the signal) and/or via one or more sensors of the device.

In some embodiments, a tachycardia episode is detected (e.g. by the device electrodes) when the measured or estimated heart rate is above 180 BPM, above 200 BPM, above 230 BPM or intermediate, higher or lower threshold. In response, ATP pulses are delivered for treating the tachycardia episode.

In some embodiments, the device is implanted outside the heart, for example in the subclavian area, and the one or more leads of the device extend into the heart, where the electrode(s) of the leads are placed in contact with cardiac tissue. Additionally or alternatively, in some embodiments, one or more leads of the device are positioned outside the heart, for example, electrodes are located in the pericardium space.

In some embodiments, the device is a combined function device configured for delivery of multiple signal types. In some embodiments, the device is configured for generating and delivering one or more of: ATP pulses, bi ventricular pacing pulses, cardiac contractility stimulations, defibrillation shocks. The different signal types may vary from each other in amplitude, duration, timing of the signal relative to the cardiac cycle, frequency, number of stimulations required, etc.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

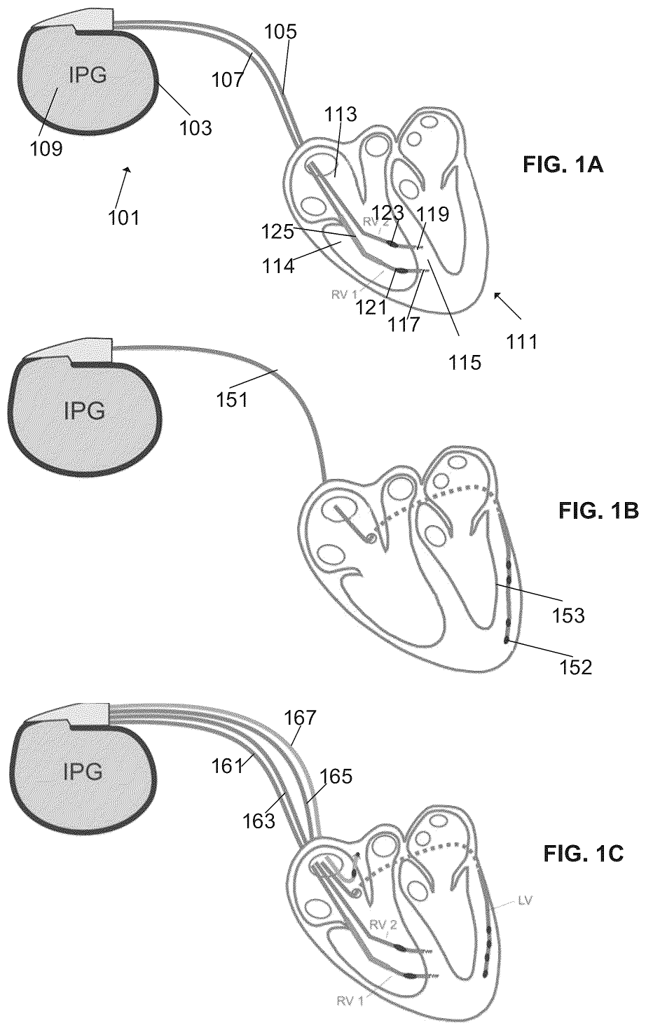

Referring now to the drawings, FIGS. 1A-C are schematic illustrations showing three exemplary configurations of an implantable cardiac device, according to some embodiments.

Referring to FIG. 1A, in some embodiments, implantable device 101 comprises a pulse generator 103. In some embodiments, pulse generator 103 comprises a housing 109 which encases, for example: powering means (e.g. a battery), control circuitry (e.g. a controller) configured for timing and generating the electrical pulses, sensing circuitry, communication circuitry, memory means, and/or other operational modules.

In some embodiments, one or more stimulation leads such as 105, 107 are operably connected to the pulse generator and extend externally from the housing. In some embodiments, the leads are connected to the device housing via a plurality of ports of the housing (not shown). In some embodiments, control of activation of one or more leads is via switch circuitry, for example switch circuitry of the device controller.

In some embodiments, a lead comprises one or more electrical wires, surrounded by an external insulating layer. In some embodiments, a lead is comprised of two wires, having different polarities. In some embodiments, a wire of the lead is coiled.

In some embodiments, pulse generator 103 is implanted outside the heart, for example in the subclavian area. Optionally, implantation is via a minimally invasive procedure.

In some embodiments, a housing of pulse generator 103 is implanted subcutaneously, for example in proximity of the left chest.

In some embodiments, leads 105 and 107 extend from pulse generator 103, and at least a distal segment of the leads is implanted within the heart 111. In some embodiments, as shown, both leads pass through the right atrium 113, into the right ventricle 114, and contact, at their distal ends, the ventricular septum 115. In some embodiments, each lead contacts the septum at a different location.

In some embodiments, each of the leads ends with a tip electrode (see 117 of lead 107, 119 of lead 105). The tip electrode may be configured as a contact electrode, a screw-in electrode, a sutured electrode, a free-floating electrode and/or other types.

In some embodiments, one or both of the leads includes a ring electrode (see 121 of lead 107, 123 of lead 105), located along the lead, proximally to the tip electrode.

In some embodiments, the ring and/or tip electrodes are implanted in the right ventricle or in the right atria of the heart.

In some embodiments, a tip electrode is formed with a threading so as to be threaded into the tissue. Alternatively, a tip electrode is solely placed in contact with the tissue.

In some embodiments, one or both of the leads include a defibrillation coil (see 125 of lead 107). Optionally, coil 125 is located along the lead, proximally to the tip electrode and/or proximally to the ring electrode.

In some embodiments, the coil is implanted in the right ventricle, right atria or in the vena cava.

In some embodiments, the tip electrodes of the leads anchor to the tissue of the ventricular septum. This may potentially improve contact with the tissue and reduce undesired movement and/or detachment of the leads.

Referring to FIG. 1B, in some embodiments, the implantable device comprises a single lead 151 which includes a plurality of spaced apart electrodes 152 (e.g. 2, 3, 4, 5, 6, 8 or intermediate, larger or smaller amount of electrodes). Optionally, lead 151 is introduced to the left ventricle 153, for example through the coronary sinus. Additionally or alternatively, a single lead having a plurality of electrodes is introduced to the right ventricle.

Referring to FIG. 1C, in some embodiments, the implantable device comprises multiple leads including, for example: right ventricle leads 161, 163; a left ventricle lead 165; and optionally a lead 167 which is introduced to the right atrium.

It is noted that one or more leads may be positioned in other locations, with consequently different effect circles and/or targeting different tissues.

In some embodiments, a lead is implanted outside the heart, and one or more additional leads are implanted inside the heart.

In some embodiments, one or more lead electrodes are used as sensors, for example for measuring an R wave amplitude and/or an RR interval of the cardiac cycle.

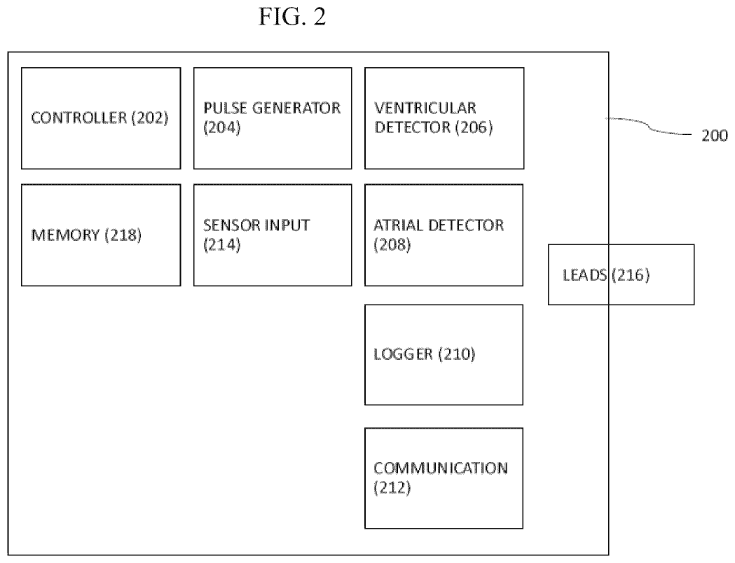

FIG. 2 is a block diagram of components of an implantable cardiac device, according to some embodiments.

In some embodiments, device 200 includes one or more leads 216 (optionally two leads), which are optionally coupled to device 200 (such as to the device housing), for example via one or more respective connectors (not shown).

In some embodiments, a pulse generator 204 is configured to generate a signal, optionally upon command form a controller 202. In some embodiments, the pulse generator includes powering circuitry, for example, including one or more capacitors. In some embodiments, the pulse generator comprises or is operably connected to a power source, such as a battery.

In some embodiments, a ventricular detector 206 is provided and used to detect atypical ventricular activation, such as ventricular tachycardia.

In some embodiments, an atrial detector 208 is provided and used to detect atypical atrial activation, such as atrial fibrillation, atrial flutter.

In some embodiments, a sensor input 214 may receive data from one or more sensors, for example electrical sensors or other sensors, such as flow, pressure and/or acceleration sensors. Optionally, one or more device electrodes are used as sensors, in addition or alternatively to delivering a signal to the tissue. In some embodiments, data obtained by the sensors is processed (e.g., by controller 202 and/or by detectors 206, 208) and optionally used as input to decision making processes in device 200. For example, in some embodiments, ATP pulse signal parameters (e.g. amplitude, width, interval between pulses, duration of pulse train, and/or other parameters) are selected in response to data acquired by the device sensors (optionally, the device electrodes).

In some embodiments, controller 202 is configured to execute one or more logics to decide, for example, signal parameters such as timing for applying the signal, an amplitude of the signal, a duration of the signal.

In some embodiments, the controller commands the applying of pulses in response to data received from the one or more sensors. For example, upon detection of a rise in heart rate (optionally above a set threshold), the controller commands the applying of pacing pulses.

In some embodiments, the controller commands the applying of pulses according to a treatment plan. Optionally, the controller effects a change in the plan, for example so as to compensate for real time deviations from the treatment plan (e.g. a skipped stimulation). In some embodiments, the controller generates commands for electrifying one or more leads of the device with a signal. Optionally, commands are generated according to the treatment plan. In some embodiments, upon a command generated by the controller, electrical current is conducted via the one or more leads and optionally to tissue contacted by the one or more leads.

In some embodiments, a memory 218 is optionally provided, for example, to store logic, past effects, therapeutic plan, adverse events and/or pulse parameters.

In some embodiments, the controller and/or memory are programmed with one or more treatment plans (optionally set for the specific patient) and/or with one or more fallback treatment plans.

In some embodiments, the controller refers to a look up table, database or the like which ties between specific cardiac events (e.g. a sensed tachycardia episode) and instructions for treating that event.

In some embodiments, a logger 210 is optionally provided to store activities of device 200 and/or of the patient. Such a log and/or programming may use a communication module 212 to send data from device 200, for example, to a programmer, a physician, a hospital or clinic database. In some embodiments, the communication module is configured to receive data, for example, pulse parameters to be programmed into the device.

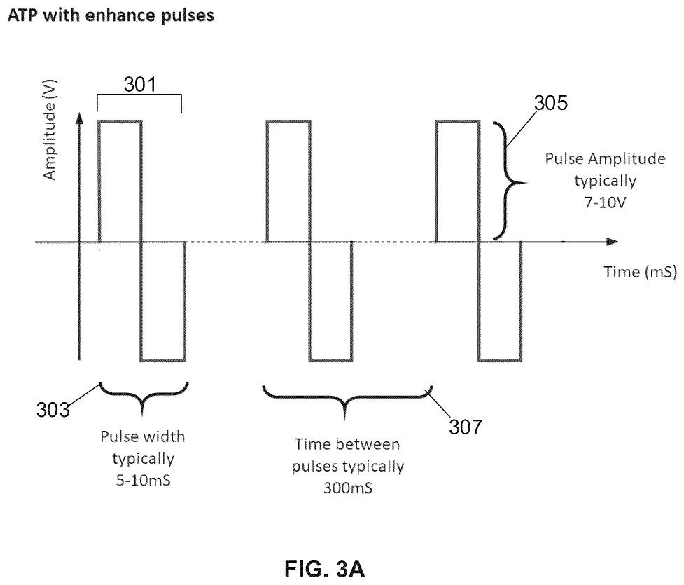

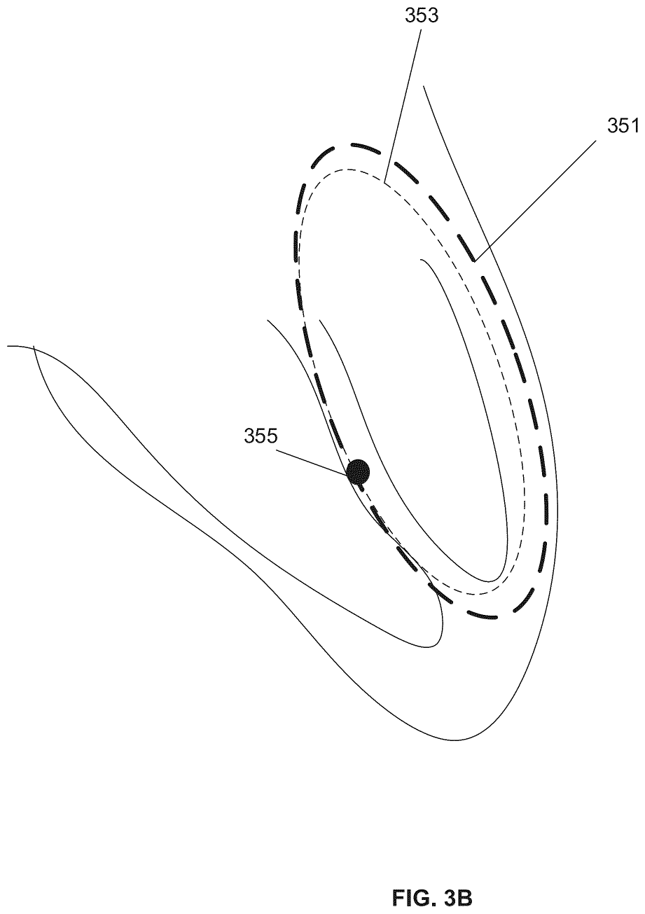

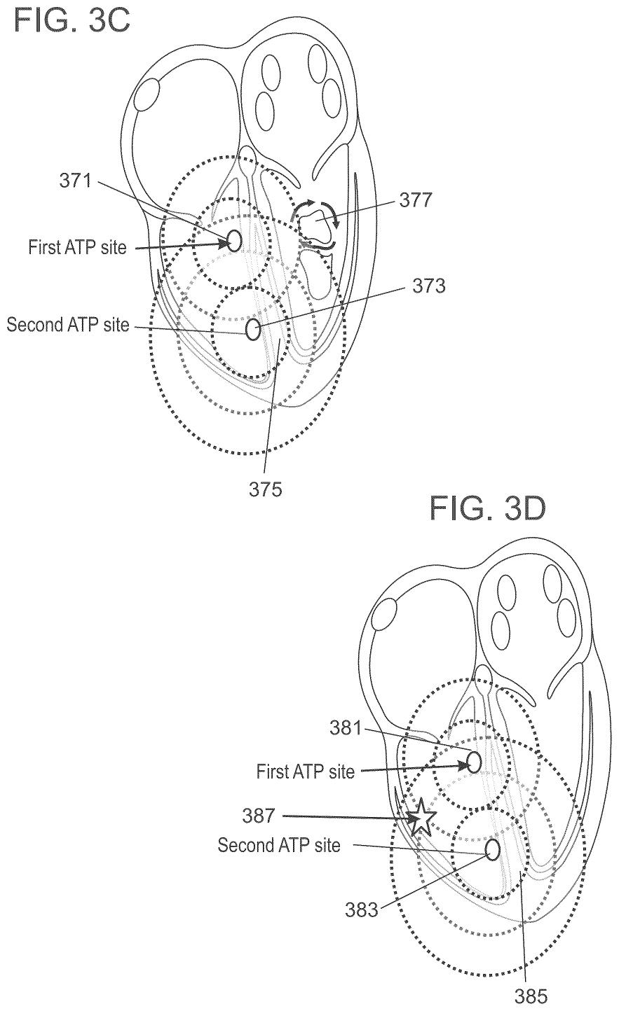

FIG. 3A is a diagram demonstrating anti tachycardia pacing, according to some embodiments.

In some embodiments, an implanted cardiac device for example as described herein is configured to generate and deliver anti-tachycardia pacing pulses 301. In some embodiments, as shown, the pulses are delivered as a biphasic waveform. In some embodiments, a train of pulses is delivered. In some embodiments, the pulse train comprises a rectangular or squared waveform.

In some embodiments, a width (or duration) 303 of a single pacing pulse 301 is between 5-10 msec. 3-7 msec, 5-15 msec or intermediate, longer or shorter. Optionally, the pulse width is higher than 2 msec, 4 msec, 6 msec or intermediate, longer or shorter.

In some embodiments, an amplitude 305 of a single pacing pulse 301 is between 7-10 Volt, 5-8 Volt, 3-12 V, or intermediate, higher or lower amplitude. Optionally, the pulse amplitude is higher than 7 Volt, higher than 8 Volt, higher than 9 Volt or intermediate, higher or lower.

In some embodiments, a time interval 307 between consecutive pulses (i.e. between initiations of consecutive pulses) is between 200-500 msec, for example 250 msec, 300 msec, 400 msec or intermediate, shorter or longer interval.

In some embodiments, a pulse width X pulse amplitude value is greater than 15 Volt*millisecond, 25 Volt*msec, 50 Volt*msec, 70 Volt*msec or intermediate, larger or smaller. Optionally, the pulse width X pulse amplitude is between 35-100 Volt*msec, between 50-75 Volt*msec, between 20-60 Volt*msec or intermediate, higher or lower.

In some embodiments, a cardiac device configured for applying anti-tachycardia pacing pulses having an amplitude of, for example, between 7.5V-10V is provided with powering means and circuitry suitable for generating pulses of this relatively high voltage.

FIG. 3B schematically illustrates a potential effect of anti-tachycardia pacing pulses on cardiac tissue, according to some embodiments.

In some embodiments, ATP pulses are delivered via at least one electrode 355, which contacts tissue located inside the heart. In this example, electrode 355 is located in contact with the ventricular septum. Additional intra-cardiac locations in which the electrode may be positioned include the right ventricle walls, the left ventricle walls, the cardiac septum. In some embodiments, one or more electrodes are located in the right and/or left atrium.

In some embodiments, pacing pulses are delivered to treat ventricular tachycardia, by providing a stimulation having parameters suitable to interrupt the arrhythmia and restore a normal sinus rhythm. In some embodiments, pacing pulses are timed to break the re-entrant circuit, for example by pacing at a higher rate than the tachyarrhythmia heart rate, so as to “take over” the cycle.

In some embodiments, the ATP pulses are delivered over a time period short enough to reduce or prevent a risk of acceleration of the existing arrhythmia.

In some embodiments, the ATP pulses delivered are wider (longer) and/or stronger (have a higher amplitude) than common ATP pulses known in the art, for example, pulses having a width of between 0.5-2 msec each.

For example, the delivered pulse is at least 50%, at least 80%, at least 100%, at least 120% or intermediate, larger or smaller percentage longer than a length (duration) of a commonly used ATP pulse. A potential advantage of enhanced (wider and/or stronger) ATP pulses may include affecting a larger area of cardiac tissue (as schematically indicated by 351) for example as compared to the tissue area affected by standard (known in the art) ATP pulses (as schematically indicated by 353). Optionally, the enhanced pulse affects a larger number of myocardial cells, for example, signaling their contraction. This may allow for interrupting the arrhythmia faster and/or with a lesser amount of ATP pulses delivered, for example as compared to standard ATP.

Another potential advantage of a stronger and/or longer pulse may include improved conduction of the excitatory signal, for example since the longer and/or stronger signal may be effective to capture myocardial cells located adjacent damaged tissue regions, where the signal conduction could be slowed down or the signal would be caused to pass along an alternative conduction pathway.

In some embodiments, a field of effect of the stronger and/or longer ATP pulse on tissue affects purkinje fibers extending along the left ventricle wall and the right ventricle wall. Direct excitation of the purkinje fibers by the ATP pulse may improve conduction and activation of the ventricle wall tissue.

FIGS. 3C-D schematically illustrate a field of effect of ATP pulses on cardiac tissue in two examples of ventricular tachycardia conditions, according to some embodiments.

FIG. 3C schematically shows an effect of ATP pulses delivered to two intra-cardiac locations, for example via two electrodes 371, 373. In some embodiments, as shown in this example, the electrodes are located in contact with tissue of the ventricular septum 375.

In some cases, ventricular tachycardia involves a re-entry cycle which occurs and/or is caused by tissue in which conduction is impaired, for example damaged or scarred tissue 377. In such cases, the action potential may travel around the damaged or scarred tissue and cause a new (or renewed) activation of the cells, potentially leading to tachycardia.

In some embodiments, for treating the tachycardia, ATP pulses are applied via the at least two electrodes. A potential advantage of applying ATP pulses at two more intra-cardiac locations as shown, optionally in a synchronized manner, may include suppressing the re-entry cycle more effectively, for example since the action potentials generated by the applied ATP pulses travels from the two electrode locations and affects a larger portion of tissue, optionally simultaneously, thereby “taking over” the re-entry cycle.

FIG. 3D schematically shows an effect of ATP pulses delivered to two intra-cardiac locations, for example via two electrodes 381, 383. In some embodiments, as shown in this example, the electrodes are located in contact with tissue of the ventricular septum 385. In some cases, ventricular tachycardia results from a focal arrhythmogenic site 387, which acts as a pacemaker which activates contraction at a high rate.

In some embodiments, for treating the tachycardia, ATP pulses are applied via the at least two electrodes. A potential advantage of applying ATP pulses at two more intra-cardiac locations as shown, optionally in a synchronized manner, may include suppressing the focal arrhythmogenic site, and “capturing” additional tissue regions in other portions of the heart, thereby potentially reducing the effect of the focal arrhythmogenic site.

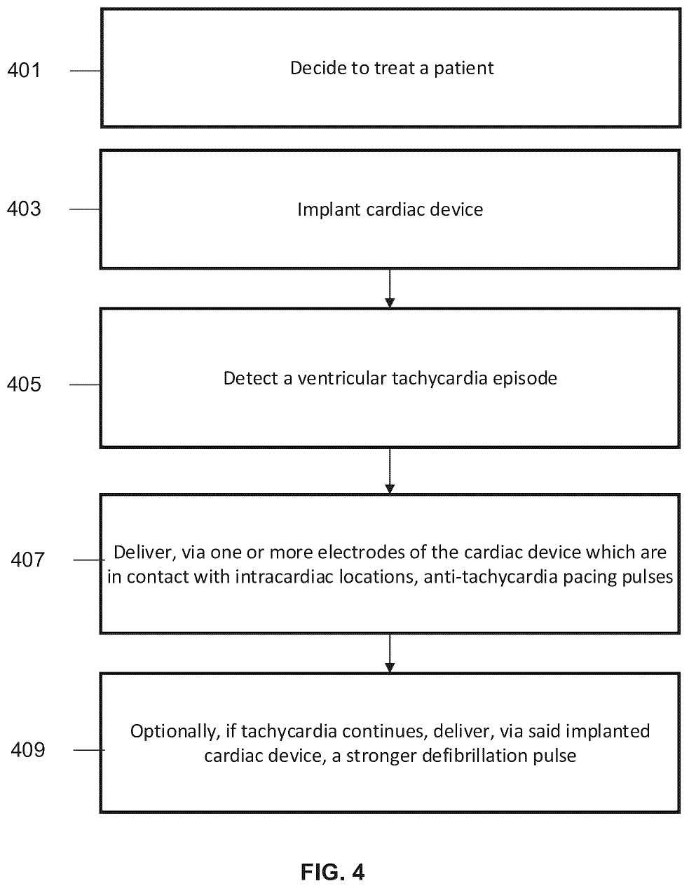

FIG. 4 is a flowchart of a general method for delivering via one or more intra-cardiac electrodes enhanced pacing pulses for treating tachycardia, according to some embodiments.

In some embodiments, a decision is made (e.g. by a physician) to treat a patient (401). In some embodiments, a patient selected for treatment is a patient suffering from heart rhythm disorders, for example, tachycardia. In some embodiments, a patient suffering from cardiac conditions such as heart failure, congestive heart failure, and/or like symptoms is selected for treatment.

In some embodiments, a cardiac device configured for pacing the heart for example by ATP is implanted in the patient (403). In some embodiments, the device comprises a pulse generator which is optionally implanted outside the heart, for example in the subclavian area, and one or more leads for delivering a signal to the heart.

In some embodiments, a ventricular tachycardia episode is detected (405). In some embodiments, ventricular tachycardia is detected when the heart rate is above a threshold, for example, above 160 BPM, above 187 BPM, above 200 BPM, above 230 BPM or intermediate, higher or lower rate. In some embodiments, a heart rate of between 187-250 BPM is indicative of tachycardia. In some embodiments, ventricular tachycardia is detected when the cardiac cycle length (heartbeat) is between 240-320 msec, 260-300 msec, 220-320 msec or intermediate, higher or lower range.

In some embodiments, the heart rate and/or cardiac cycle length is measured by the implanted device, for example by the device electrodes. In some embodiments, the heart rate and/or cardiac cycle length are calculated according to an R-R interval measured by the electrodes.

In some embodiments, the device is programmed so that upon detection of a tachycardia episode, ATP pulses are delivered via one or more device electrodes which are in contact with intra-cardiac locations (407), for example, in contact with myocardial tissue.

In some embodiments, two electrodes contact two different intra-cardiac locations. In some embodiments, the electrodes contact at least two different locations along the ventricular septum. In some embodiments, the electrodes contact at least two different locations of the left ventricular wall. In some embodiments, the electrodes contact at least two different locations of the right ventricular wall. In some embodiments, one electrode contacts the left ventricular wall and another contacts the right ventricular wall. In some embodiments, the electrodes contact at least two different locations in the right side of the ventricular septum.

A potential advantage of delivering ATP pulses to at least two tissue locations may include increasing a likelihood of interrupting the tachycardia reentry. Optionally, the tachycardia is interrupted as a result of affecting, simultaneously, tissue areas at the two locations contacted by the electrodes. Optionally, the tachycardia is interrupted as a result of the pacing pulse being applied to the at least two locations in an order (e.g. with a time interval) that is determined according to a natural propagation direction and/or speed of the action potential.

In some cases, the reentry focal point is located at or adjacent scarred or damaged myocardial tissue. Optionally, one or more electrodes of the cardiac device may be positioned adjacent or at a suspected reentry focal point.

In some embodiments, the ATP pulses are delivered with parameters for example as described hereinabove in FIG. 3A.

In some embodiments, a combination of pulses is delivered where some pulses have a higher amplitude and/or a longer duration than others. Optionally, pulses are delivered in pairs, for example, each pair delivered within a single cardiac cycle. In an example, a short pulse is provided first and a longer pulse is delivered second, for example during a refractory period triggered by the first pulse.

In some embodiments, pulses are gradually increased in duration and/or amplitude.

In some embodiments, if tachycardia continues despite the applied ATP pulses, a stronger defibrillation pulse is delivered (409). In some embodiments, defibrillation pulse(s) are delivered after a set number of cardiac cycles during which ATP pulses were applied, for example, after 2 cycles, after 4 cycles, after 6 cycles, after 10 cycles or intermediate, larger or smaller number. In some embodiments, the defibrillation pulse energy is between 10-100 Joules, for example, 30 Joules, 50 Joules, 25 Joules or intermediate, higher or lower energy.

A potential advantage of delivering ATP pulses for targeting ventricular tachycardia as opposed to directly applying a defibrillation pulse may include reducing or preventing damage to cardiac tissue which may occur as a result of the strong electrical shock delivered by the defibrillation pulse.

A potential advantage of ATP pulses having an amplitude for example as described herein (e.g. 7-10 V) may include improving the ability of the stimulation to take over the tachycardia rhythm, potentially reducing a need for delivering a defibrillation pulse.

The following is an example of an operation scheme for an implanted cardiac device configured for delivering a plurality of cardiac stimulation signals, including, but not limited to: pacing signals (e.g. ventricular pacing signals), defibrillation signals, cardiac contractility modulation signals, and/or anti-tachycardia pacing signals. In an example, the implantable device is configured for generating and delivering all of the following stimulation types: bi-ventricular pacing, ATP pulses, cardiac contractility modulation signals, defibrillation shocks.

Exemplary Operation Scheme of an Implantable Device Configured for Combined CRT, CCM, ICD and/or ATP Treatment:

In some embodiments, when BPM is within a normal range, the device delivers bi-ventricular pacing (CRT cardiac resynchronization therapy).

Optionally, during part of the time and/or at predefined timings, the device delivers cardiac contractility modulation (CCM) stimulations, such as non-excitatory stimulations. In some embodiments, CCM stimulations are delivered during normal sinus rhythm. In some embodiments, CCM stimulations have an amplitude between 7-10 V and a duration between 4-6 msec. The cardiac contractility modulation stimulations are optionally delivered during the ventricle refractory period, for example, 0.5-5 msec following CRT pacing. In some embodiments, the CCM stimulation is delivered via one or more leads contacting the ventricular septum.

In some embodiments, the cardiac contractility modulation stimulation is selected to increase the contractility of a cardiac ventricle when the electric field of the signal stimulates such ventricular tissue, for example, the left ventricle, the right ventricle and/or a ventricular septum. In some embodiments, contractility modulation is provided by phosphorylation of phospholamban caused by the signal. In some embodiments of the invention, contractility modulation is caused by a change in protein transcription and/or mRNA creation caused by the signal, optionally in the form of reversal of a fetal gene program.

In some embodiments, if a BPM above a certain threshold is detected by the device, for example BPM>180, BPM>200, BPM>160 or intermediate, higher or lower threshold, ATP pulse signals having parameters for example as described herein are delivered by the device.

In some embodiments, if the BPM continues to be high over a certain time period (e.g over a pre-defined time period, for example as programmed into the device) and/or if the BPM as initially detected is higher than a second threshold, for example BPM>250, BPM>300, BPM>280, BPM>320 or intermediate higher or lower threshold, a defibrillation shock is delivered by the device.

In some embodiments, a determination between which type of signal to apply, which signal parameters to select, and when to time the signal is made by the device controller, for example in response to measurements obtained by the one or more electrodes of the device and/or by one or more sensors of the device and/or one or more sensors external to the device.

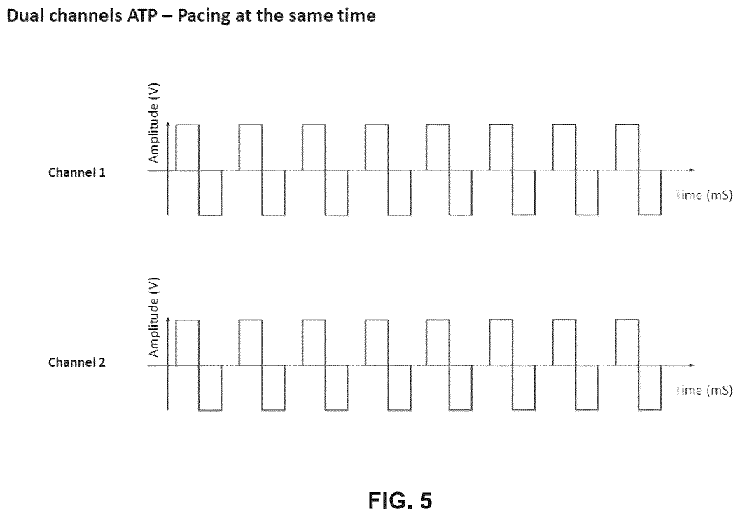

FIG. 5 is a diagram demonstrating anti tachycardia pacing by delivering synchronized pacing pulses to at least two different cardiac locations, according to some embodiments.

In some embodiments, ATP pulses are delivered to at least two spaced apart cardiac locations, for example via at least two device electrodes. In some embodiments, the electrodes contact at least two different locations along the ventricular septum. In some embodiments, the electrodes contact at least two different locations of the left ventricular wall. In some embodiments, the electrodes contact at least two different locations of the right ventricular wall.

In some embodiments, one electrode contacts the left ventricular wall and another contacts the right ventricular wall. In some embodiments, a plurality of electrodes are in contact with right ventricle tissue and a plurality of electrodes are in contact with left ventricle tissue.

In some embodiments, parameters of the signal delivered via each of the two electrodes (indicated as “channel 1” and “channel 2”) in the figure are as described, for example, for FIG. 3A above. It is noted that the signal parameters are not limited to those described. Additional signal parameters for pacing pulses delivered to two or more locations may include, for example, an amplitude of between 1-20 V, 5-15 V, 8-13 V, or intermediate, higher or lower amplitude; and, for example, a width of between 0.1-20 msec, 1-10 msec, 5-15 msec or intermediate, longer or shorter width.

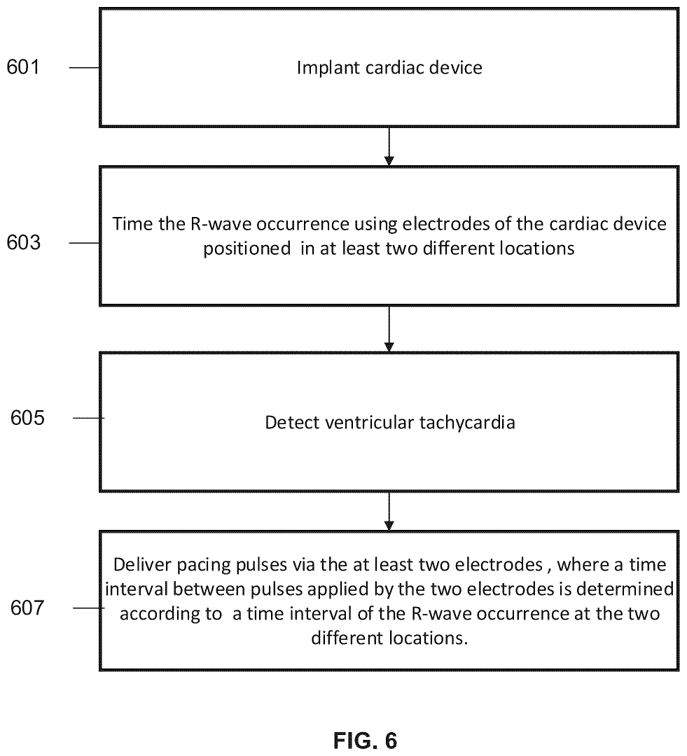

FIG. 6 is a flowchart of a method for timing anti tachycardia pacing delivered to at least two different cardiac locations according to a measured time interval of R-wave occurrence at the two locations, according to some embodiments.

In some embodiments, a cardiac device for example as described herein is implanted (601).

In some embodiments, electrical activity of the heart is measured and/or estimated, for example during normal sinus rhythm. In some embodiments, an R-wave occurrence is timed (603), optionally via device electrodes positioned in at least two different cardiac locations. In some embodiments, by timing the R-wave occurrence, propagation of the natural electrical signal can be estimated by determining the time interval of the R-wave occurrence between the two different locations.

In some embodiments, a ventricular tachycardia episode is detected (605), for example when the measured heart rate is above a threshold, such as higher than 160 BPM, above 187 BPM, above 200 BPM, above 230 BPM or intermediate, higher or lower rate.

In some embodiments, ATP pulses delivered via the at least two electrodes to the at least two different cardiac locations are timed according to the measured interval of R-wave occurrence (607).

In some embodiments, an order in which ATP pulses are delivered via the at least two electrodes to the at least two cardiac locations is set to match a natural propagation direction of the action potential, for example as occurring during normal cardiac function.

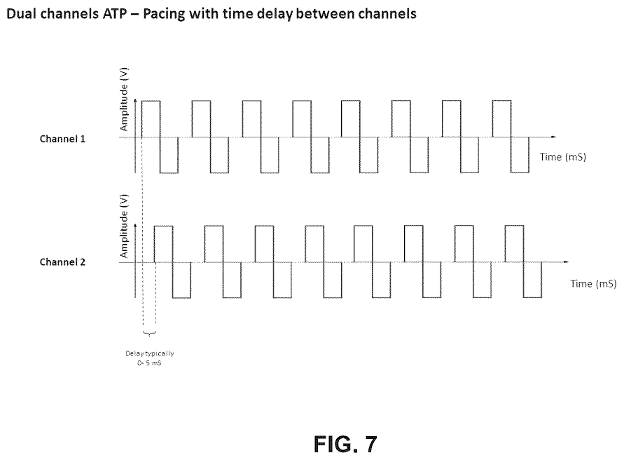

FIG. 7 is a diagram demonstrating anti tachycardia pacing by delivering to at least two different cardiac locations pacing pulses timed according to a time interval of R-wave occurrence at the two locations.

In the example shown, an ATP signal delivered via the 2nd channel is provided at a delay of between 0.1-5 msec, 2-4 msec, 1-6 msec, or intermediate, longer or shorter time period from the ATP signal delivered via the 1st channel.

In some embodiments, a frequency of the ATP signal (for example of the signal delivered via each of the channels) is selected to be high enough for overriding the tachyarrhythmia episode and restoring normal sinus rhythm. In some embodiments, the ATP pulse rate is between 150-300 BPM, 200-600 BPM, 180-240 BPM or intermediate, higher or lower rate.

In some embodiments, pulses are delivered to multiple cardiac sites (e.g. 2, 3, 4, 6, or intermediate, higher or lower number of sites) according to a defined order, which is optionally pre-programmed into the device controller. Optionally, the order of delivering to the different sites is set according to the anatomic propagation of the signal throughout the cardiac tissue.

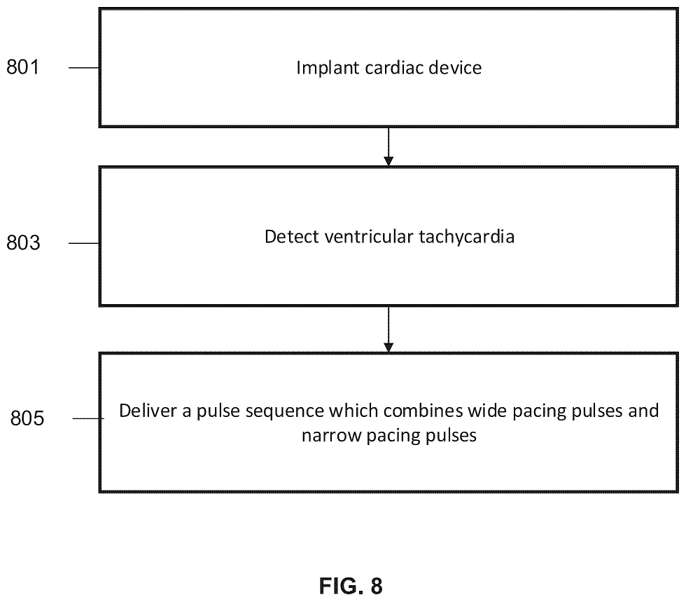

FIG. 8 is a flowchart of a method for delivering a pulse sequence which combines wide pacing pulses and narrow pacing pulses for treating tachycardia, according to some embodiments.

In some embodiments, a cardiac device for example as described herein is implanted (801).

In some embodiments, upon detection of a ventricular tachycardia episode (803), an ATP pulse sequence is delivered by the implanted device, including a combination of wide pacing pulses (pulses that are longer in duration) and narrow pulses (pulses that are shorter in duration) (805).

In some embodiments, the wide pacing pulses are each of between 2-10 msec long, such as 3 msec, 5 msec. 8 msec or intermediate, longer or shorter duration. In some embodiments, the narrow pacing pulses are each of between 0.2-2 msec long, such as 0.5 msec, 1 msec, 1.5 msec or intermediate, longer or shorter pulse duration.

In some embodiments, an amplitude of each pacing pulse is between 7-10 Volt, 5-8 Volt, 3-12 V, or intermediate, higher or lower amplitude. Optionally, the pulse amplitude is higher than 7 Volt, higher than 8 Volt, higher than 9 Volt or intermediate, higher or lower.

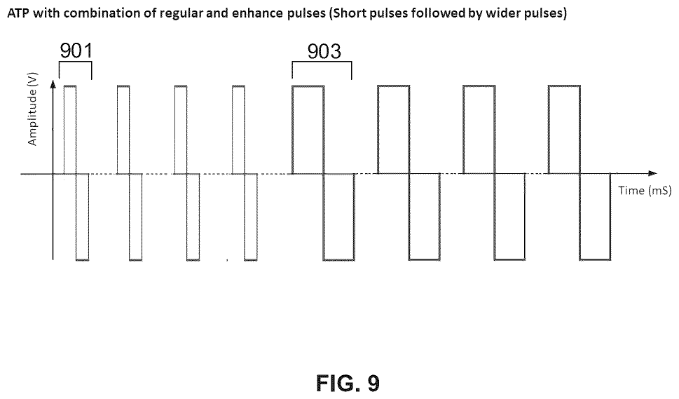

Various examples of ATP pulse sequences combining pulses that vary in duration are shown in FIGS. 9-12.

In the example of FIG. 9, the ATP pulse sequence includes a plurality of bi-phasic narrow pulses 901 (e.g. 1, 4, 6, 8, 10, 16, 20 pulses or intermediate, larger or smaller number) followed by a plurality of wide pulses 903 (e.g. 1, 4, 6, 8, 10, 16, 20 pulses or intermediate, larger or smaller number).

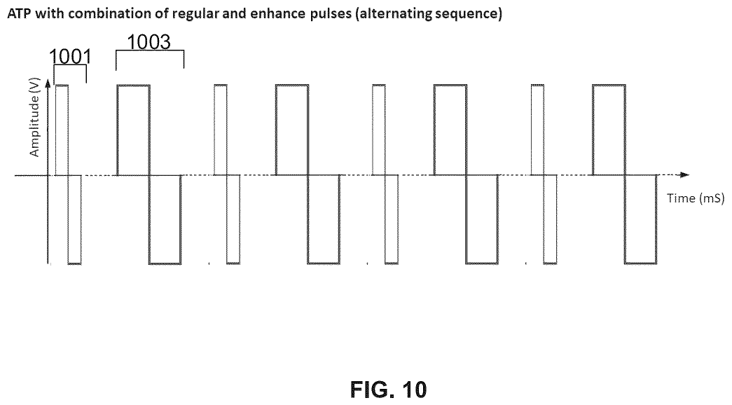

In the example of FIG. 10, the ATP pulse sequence includes an alternating pattern in which a bi-phasic narrow pulse 1001 is followed by a bi-phasic wide pulse 1003 and so forth.

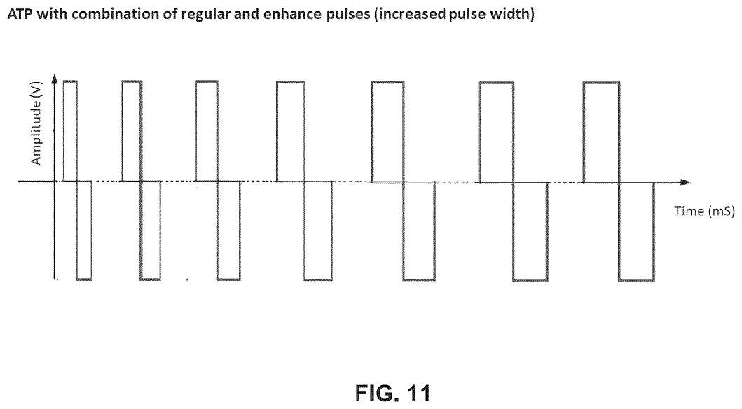

In the example of FIG. 11, the ATP pulse sequence includes bi-phasic pulses which increase in duration over time. Optionally, the pulse duration increases in constant intervals, such as 0.5 msec, 1 msec, 1.5 msec, 2 msec or intermediate, longer or shorter.

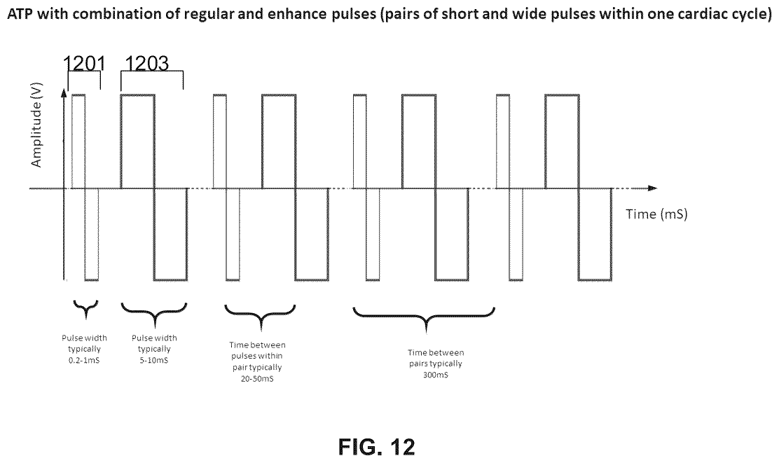

In the example of FIG. 12, the ATP pulse sequence includes consecutive pairs where each pair is comprised of a narrow pulse 1201 (e.g. 0.2-1 msec long) and a wide pulse 1203 (e.g. 5-10 msec long). In some embodiments, each pair is delivered during a single cardiac cycle. In some embodiments, the wide pulse of the pair is timed to be delivered during a refractory period triggered by the preceding narrow pulse.

Alternatively, in some embodiments, each pair includes a wide pulse followed by a narrow pulse.

Optionally, a time interval between initiation of the narrow pulse and initiation of the wide pulse of each pair is between 20-50 msec, between 30-40 msec, between 10-30 msec, or intermediate, longer or shorter interval. Optionally, a time interval between sequential pairs (such as between initiation of a first pair and initiation of a second, sequential pair) is between 200-400 msec. 100-300 msec, 250-350 msec, or intermediate, longer or shorter.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An implantable cardiac device comprising:
at least one lead electrode configured to contact intra-cardiac tissue;
a housing including circuitry for controlling and activating said at least one lead electrode; and
a pulse generator configured to generate anti-tachycardia pacing pulses to be delivered by said at least one lead electrode, wherein an anti-tachycardia pacing pulse comprises at least one of: a duration of between 5-10 msec and an amplitude of between 5-10V; and
wherein said pulse generator is configured to generate synchronized anti-tachycardia pacing pulses to be delivered via at least two lead electrodes configured to be positioned in at least two different intra-cardiac locations.

2. The device according to claim 1, wherein said at least one lead electrode is configured to contact said intra-cardiac tissue located in one of the following: the ventricular septum, the left ventricle wall, the right ventricle wall.

3. (canceled)

4. The device according to claim 31, wherein said at least two lead electrodes are configured to be positioned in at least two different intra-cardiac locations from the following:
a. within the right ventricle;
b. along the ventricular septum;
c. the right ventricle and the left ventricle;
wherein said pulse generator is configured to deliver a defibrillation pulse if a detected tachycardia episode continues following delivery of said anti-tachycardia pacing pulses; wherein said circuitry is configured to detect said tachycardia episode by measuring an R-R interval via said at least one lead electrode; wherein said circuitry is configured to detect said tachycardia episode when measuring a heart rate of between 180-250 BPM; wherein said anti-tachycardia pacing pulse is a biphasic pulse; wherein said circuitry comprises a memory which stores a plurality of anti-tachycardia pacing pulse sequences, and a controller configured to implement the delivery of a selected anti-tachycardia pacing pulse sequence; wherein said cardiac device is further configured for one or more of:
delivering, during normal sinus rhythm, cardiac contractility modulation stimulations having a duration of between 5-10 msec and an amplitude of between 5-10V; and
delivering defibrillation shocks.

5-11. (canceled)

12. The device according to claim 1, wherein said pulse generator is configured to generate said anti-tachycardia pacing pulses simultaneously.

13. The device according to claim 1, wherein said pulse generator is configured to generate said synchronized anti-tachycardia pacing pulses with a time delay between them such that pulses applied via one of said two lead electrodes are delivered in a predetermined time delay from pulses applied via a second electrode of said at least two lead electrodes.

14. The device according to claim 13, wherein said predetermined time delay is selected according to a time difference between R-wave occurrences measured via said at least two electrodes at said at least two different cardiac locations during normal cardiac function.

15-16. (canceled)

17. The device according to claim 1, wherein said pulse generator is configured to generate anti-tachycardia pacing pulses having different durations selected from between 5-10 msec.

18. A method of anti-tachycardia pacing via an implanted cardiac device, comprising:
positioning at least one electrode of the implanted cardiac device at an intra-cardiac location;
detecting a tachycardia episode;
delivering, via said at least one electrode, anti-tachycardia pacing pulses, wherein an anti-tachycardia pacing pulse comprises at least one of: a duration of between 5-10 msec and an amplitude of between 5-10V.

19. The method according to claim 18, wherein said intra-cardiac location is one of: the ventricular septum, the left ventricle wall, the right ventricle wall; wherein said method comprising positioning at least two electrodes in at least two different intra-cardiac locations from the following:
a. within the right ventricle;
b. at two different locations along the ventricular septum;
c. at least one electrode in the right ventricle and at least one electrode in the left ventricle.

20-23. (canceled)

24. The method according to claim 18, further comprising delivering a defibrillation pulse if the tachycardia episode continues following said delivering of anti-tachycardia pacing pulses; wherein said detecting is by measuring an R-R interval via said at least one electrode of said implanted cardiac device; and wherein said anti-tachycardia pulse is a biphasic pulse.

25-26. (canceled)

27. The method according to claim 19, wherein said delivering comprises delivering synchronized anti-tachycardia pacing pulses via said at least two electrodes.

28. The method according to claim 27, wherein said delivering comprises delivering said anti-tachycardia pacing pulses simultaneously via said at least two electrodes.

29. The method according to claim 27, wherein said delivering comprises delivering said anti-tachycardia pacing pulses with a time delay such that pulses applied via one of said two electrodes are delivered in a predetermined time delay from pulses applied via a second electrode of said two electrodes.

30. The method according to claim 29, wherein said predetermined time delay is selected according to a time difference between R-wave occurrences measured by said at least two electrodes at said at least two different cardiac locations during normal cardiac function.

31. (canceled)

32. A method of anti-tachycardia pacing via an implanted cardiac device, comprising:
detecting a tachycardia episode;
delivering a pulse sequence which combines anti-tachycardia pacing pulses having different durations.

33. The method according to claim 32, wherein a pulse duration is from between 0.1-10 msec.

34. The method according to claim 32, wherein said anti-tachycardia pacing pulses include wide pacing pulses and narrow pacing pulses, wherein a wide pacing pulse comprises a duration of between 2-10 msec and a narrow pacing pulse comprises a duration of between 0.1-5 msec; and wherein said delivering comprises one or more of the following:
a. delivering a plurality of narrow pacing pulses followed by a plurality of said wide pacing pulses;
b. delivering a sequence including pairs of pulses, wherein in each pair a narrow pacing pulse is followed by a wide pacing pulse, said narrow pacing pulse and said wide pacing pulse delivered within the same cardiac cycle; and wherein said wide pacing pulse is delivered during a refractory period triggered by said narrow pulse.

35-37. (canceled)

38. The method according to claim 32, wherein said delivering comprises one or more of the following:
a. delivering a sequence of pacing pulses which gradually increase in duration; and wherein each pulse is at least 10% longer than its preceding pulse;
b. delivering a sequence of pacing pulses which gradually decrease in duration; and wherein each pulse is at least 10% shorter than its preceding pulse.

39-41. (canceled)

42. A method of anti-tachycardia pacing via an implanted cardiac device, comprising:
detecting a tachycardia episode;
delivering, via at least two electrodes positioned in contact with tissue in at least two different intra-cardiac locations, anti-tachycardia pacing pulses.

43. The method according to claim 42, further comprising measuring, during normal cardiac function, a time difference between R-wave occurrences in said at least two different intra-cardiac locations; and wherein a time interval between pacing pulses delivered to said two different intra-cardiac locations is set according to said measured time difference.

44. (canceled)
